(19) United States
(12) Patent Application Publication
(10) Pub. No.: US 2011/0130466 A1
(43) Pub. Date: Jun. 2, 2011

(54) USE OF RASAGILINE FOR THE TREATMENT OF PROGRESSIVE SUPRANUCLEAR PALSY

(76) Inventor: Stefan Lorenzl, Neuried (DE)

(21) Appl. No.: 12/901,281

(22) Filed: Oct. 8, 2010

Related U.S. Application Data
(60) Provisional application No. 61/278,677, filed on Oct. 9, 2009.

(51) Int. Cl. A61K 31/135; A61P 25/00; A61P 25/24

(52) U.S. Cl. 514/657

(57) ABSTRACT

A method for the treatment of Progressive Supranuclear Palsy. Such method includes administering to a subject an amount of R(+)-N-propargyl-1-aminoindan or a pharmaceutically acceptable salt thereof.

This application claims benefit of U.S. Provisional Application No. 61/278,677, filed Oct. 9, 2009, the contents of which are hereby incorporated by reference.

Throughout this application various publications, published patent applications, and patents are referenced. The disclosures of these documents in their entireties are hereby incorporated by reference into this application in order to more fully describe the state of the art to which this invention pertains.

### BACKGROUND

Progressive Supranuclear Palsy (PSP) is a rapidly progressing disease with a median disease duration of 6 to 7 years, characterized by early falls (tendency to topple backwards), vertical ophthalmoparesis, akinetic-rigid features, prominent bulbar dysfunction and fronto-subcortical dementia. The loss of independent gait, the inability to stand unassisted occurs less than 5 years after disease onset (Goetz C G, Leurgans S, Lang A E, Litvan I., (Mar. 25, 2003) “Progression of gait, speech and swallowing deficits in progressive supranuclear palsy”, Neurology, 60(6):917-22). The prevalence of PSP in Europe is 5 per 100,000. Pathologically, there is severe neuronal loss in the substantia nigra, globus pallidus, subthalamic nucleus, midbrain, and pontine reticular formation with frequent neurofibrillary tangles composed of straight tau filaments. PSP is a four-repeat tauopathy, in reference to the excessive deposition of a particular tau isoform (Burn D J, Lees A J., (October 2002) “Progressive supranuclear palsy: where are we now?”, Lancet Neurol., 1(6):359-69). In addition to the extensive and multifocal neuropathological changes there are multiple neurotransmitter abnormalities, including dopamine, acetylcholine, gamma-aminobutyric acid and the noradrenaline systems (Rajput A, Rajput A H., (2001) “Progressive supranuclear palsy: clinical features, pathophysiology and management”, Drugs Aging, 18(12):913-25, Review).

The disease has been described in 1963 by three physicians Dr. Steele, Richardson and Olschewski and has therefore originally been named as “Steele-Richardson-Olschewski-Syndrome.” However, retrospectively there have been reports about patients with PSP from the early 40 s of the 20th century. Certainly there have been patients earlier but they have not been classified as PSP.

The name “progressive supranuclear palsy” describes the main feature of the disease the progressive failure of arbitrary eye movements. The automated eye movements are described by the word “supranuclear”, since the automated eye movements are “nuclear” controlled.

The onset of the disease is usually between the age of 50-70 years. Men and women are equally affected. Many patients report initially to have a constant vertigo and balance problems or constant falls, typically backwards. The reduction of the arbitrary eye movements reduces the capability to read, climb stairs and drive motor vehicles.

Additional early symptoms which are sometimes not evident for the patient but can sometimes be detected by a patient's relatives are personality changes, for example, irritability or loss of impulse control. Some patients lose the interest in daily activities and hobbies. Even in the early phase of the disease mood changes and depression are very common.

The regions of the brain which control the eye movements are located close to the regions which control the tongue and muscles for swallowing. The speech of the patients is usually changed early in the disease (some months after onset). It is slowed and indistinctly, deeper and there are many breaks between the words. The swallowing of liquids and food is difficult as the disease progresses, which leads to life-threatening pneumonias. This is the main cause of death in advanced PSP, since these symptoms are normally absent in the early phase.

To date, there is no treatment for the disease as the negative outcomes of the vast majority of studies make it impossible to set standards. Dopamine agonists, monoamine oxidase inhibitors, and catechol-O-methyl transferase inhibitors are of no proven benefit (Warren N M, Burn D J., (February 2007) “Progressive supranuclear palsy”, Pract Neural., 7(1):16-23, Review).

### BRIEF SUMMARY OF THE INVENTION

The subject invention provides a method of treating a human subject suffering from Progressive Supranuclear Palsy, comprising administering to the subject an amount of R(+)-N-propargyl-1-aminoindan or a pharmaceutically acceptable salt thereof effective to treat the subject.

The subject invention also provides a method of alleviating a symptom of Progressive Supranuclear Palsy in a human subject afflicted with Progressive Supranuclear Palsy comprising administering to the subject an amount of R(+)-N-propargyl-1-aminoindan or a pharmaceutically acceptable salt thereof effective to alleviate the symptom of Progressive Supranuclear Palsy in the subject.

The subject invention further provides a pharmaceutical composition for use in the treatment of, or alleviation of symptoms of, Progressive Supranuclear Palsy, which comprises a therapeutically effective amount of R(+)-N-propargyl-1-aminoindan or a pharmaceutically acceptable salt thereof and a pharmaceutically acceptable carrier.

The subject invention yet further provides use of R(+)-N-propargyl-1-aminoindan or a pharmaceutically acceptable salt thereof for the treatment of, or alleviation of the symptoms of, Progressive Supranuclear Palsy.

### DESCRIPTION OF THE FIGURES

FIG. 1 shows the temporal profile of falls of patients taking the medication at least 8 months (n=12). Month 1 is the baseline.

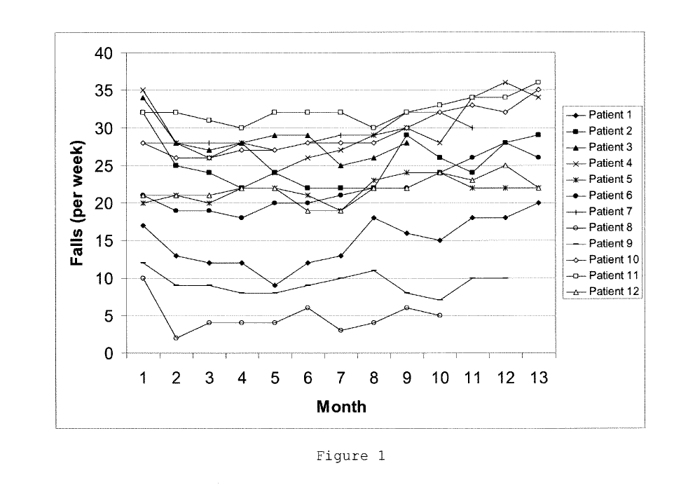

FIG. 2A shows a posturographic measurement of patient 5 before a 6 month treatment regimen with rasagiline.

FIG. 2B shows a posturographic measurement of patient 5 after a 6 month treatment regimen with rasagiline.

FIG. 3 illustrates different sway patterns of a normal person, a patient with Parkinson's disease, and a PSP patient.

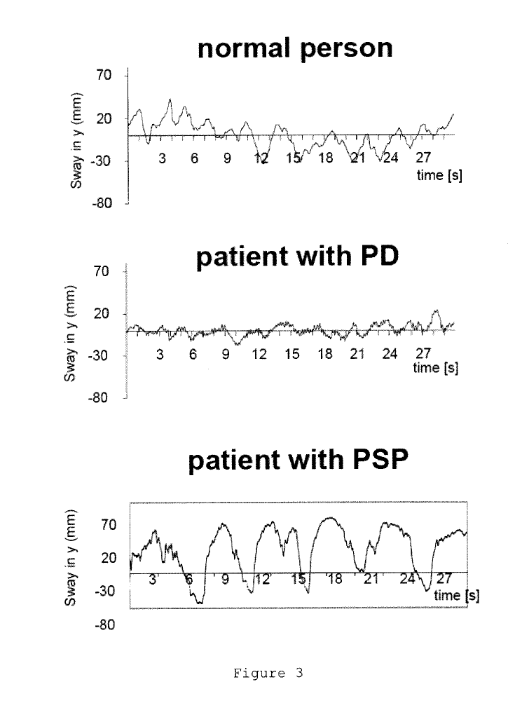

### DETAILED DESCRIPTION OF THE INVENTION

The subject invention provides a method of treating a human subject suffering from Progressive Supranuclear Palsy, comprising administering to the subject an amount of R(+)-N-propargyl-1-aminoindan or a pharmaceutically acceptable salt thereof effective to treat the subject.

The subject invention provides a method of alleviating a symptom of Progressive Supranuclear Palsy in a human subject afflicted with Progressive Supranuclear Palsy comprising administering to the subject an amount of R(+)-N-propargyl-1-aminoindan or a pharmaceutically acceptable salt thereof effective to alleviate the symptom of Progressive Supranuclear Palsy in the subject.

In an embodiment of the method, the symptom of Progressive Supranuclear Palsy is postural instability, frequent falls, visual disturbances, speech disturbances, ataxia, dysphagia, pneumonia or depression.

In another embodiment of the method, the amount of R(+)-N-propargyl-1-aminoindan or of the pharmaceutically acceptable salt thereof is from 0.01 mg to 20 mg per day.

In yet another embodiment of the method, the amount of R(+)-N-propargyl-1-aminoindan or of the pharmaceutically acceptable salt thereof is from 0.5 mg to 5 mg per day.

In yet another embodiment of the method, the amount of R(+)-N-propargyl-1-aminoindan or of the pharmaceutically acceptable salt thereof is 2 mg per day.

In yet another embodiment of the method, the amount of R(+)-N-propargyl-1-aminoindan or of the pharmaceutically acceptable salt thereof is 1 mg per day.

In yet another embodiment of the method, the amount of R(+)-N-propargyl-1-aminoindan or of the pharmaceutically acceptable salt thereof is 0.5 mg per day.

In yet another embodiment of the method, the administration is of the pharmaceutically acceptable salt of R(+)-N-propargyl-1-aminoindan.

In yet another embodiment of the method, the pharmaceutically acceptable salt is esylate, mesylate, sulphate, citrate or tartrate.

In yet another embodiment of the method, the pharmaceutically acceptable salt is mesylate.

In yet another embodiment of the method, the amount of R(+)-N-propargyl-1-aminoindan mesylate is 1.56 mg per day.

In yet another embodiment of the method, the administration is oral, parenteral, rectal or transdermal.

The subject invention also provides a pharmaceutical composition for use in the treatment of, or alleviation of symptoms of, Progressive Supranuclear Palsy, which comprises a therapeutically effective amount of R(+)-N-propargyl-1-aminoindan or a pharmaceutically acceptable salt thereof and a pharmaceutically acceptable carrier.

The subject invention further provides use of R(+)-N-propargyl-1-aminoindan or a pharmaceutically acceptable salt thereof for the treatment of, or alleviation of the symptoms of, Progressive Supranuclear Palsy.

As used herein, “a human subject suffering from Progressive Supranuclear Palsy” is a human subject who has been diagnosed with Progressive Supranuclear Palsy.

As used herein, “a human subject afflicted with Progressive Supranuclear Palsy” is a human subject who has been diagnosed with Progressive Supranuclear Palsy.

As used herein, “Posturographic measurement” is a measurement to evaluate the standing ability of a person under different conditions, e.g. with eyes closed.

As used herein, “Progressive Supranuclear Palsy Rating Scale (PSPRS)” comprises 28 items in six categories: daily activities, behaviour, bulbar, ocular motor, limb motor and gait/midline. Scores range from 0 to 100, each item graded 0-2 (six items) or 0-4 (22 items).

As used herein, NNIPPS is a clinical trial of riluzole involving nearly 800 people diagnosed with the ‘parkinson plus’ syndromes of multiple system atrophy (MSA) and progressive supranuclear plasy (PSP). In addition to showing whether riluzole is helpful in MSA and PSP, NNIPPS will improve criteria for making an accurate and early diagnosis, for assessing the rate of progression, and will advance understanding of the biology of these disabling and progressive neurodegenerative diseases.

As used herein, “Schwab and England score”, is described in Schwab R S, England A C. J, (October 1958) “Parkinson's disease”, Chronic Dis., 8(4):488-509.

As used herein, “Montgomery-Asberg Depression Rating Scale (MADRS)” is a ten-item diagnostic questionnaire which psychiatrists use to measure the severity of depressive episodes in patients with mood disorders. It was designed in 1979 by British and Swedish researchers as an adjunct to the Hamilton Rating Scale for Depression (HAMD).

As used herein, “Frontal Assessment Battery (FAB)” is a brief tool that can be used at the bedside or in a clinic setting to assist in discriminating between dementias with a frontal dysexecutive phenotype and Dementia of Alzheimer's Type (DAT). The FAB has validity in distinguishing Fronto-temporal type dementia from DAT in mildly demented patients (MMSE>24). Total score is from a maximum of 18, higher scores indicating better performance.

As used herein, Mann-Whitney U test (also called the Mann-Whitney-Wilcoxon (MWW), Wilcoxon rank-sum test, or Wilcoxon-Mann-Whitney test) is a non-parametric test for assessing whether two independent samples of observations come from the same distribution. It is one of the best-known non-parametric significance tests. It was proposed initially by Frank Wilcoxon in 1945, for equal sample sizes, and extended to arbitrary sample sizes and in other ways by H. B. Mann and Whitney (1947). MWW is virtually identical to performing an ordinary parametric two-sample t test on the data after ranking over the combined samples.

As used herein, MMSE refers to Mini-Mental State Examination.

As used herein, MPTP (1-methyl-4-phenyl-1,2,3,6-tetrahydropyridine) is a neurotoxin that causes permanent symptoms of Parkinson's disease by killing neu in the substantia nigra of the brain.

As used herein, LPLV refers to Last Patient Last Visit.

As used herein, FPFV refers to First Patient First Visit.

As used herein, FPLV refers to First Patient Last Visit.

As used herein, ECG refers to Electrocardiogram.

As used herein, difference stages of PSP are described as follows:

Phase 1—Deterioration of handwriting and difficulty writing; Speech problems, difficulty being understood by others, slurring, etc.; Coordination problems leading to unexpected falls and stumbling; Change in walking rhythms/patterns; Vision problems; Lethargy, apathy, no desire to do anything; Changes in sleep patterns; Cognitive problems; Decrease of sound judgement; Decrease in modesty; Increase in impatience and irritability.

Phase 2—Problems with sitting down or getting up; Cannot lower self into chair gently, just ‘plops’ down; Increased difficulty walking; Begins using a cane for balance, will progress to a walker; Increased number of falls; Stooped posture because of vision problems, can't see downward easily; Problems with opening or closing eyes, some patients get ‘dry eye’ because their eyes do not close all the way; Difficulty dressing, cannot do buttons or zippers because hands and fingers do not work as they used to; Almost impossible to write anything legibly; Eating problems; Coughing and choking; Loss of eating etiquette, fills mouth too full, lots of spills, begins wearing a bib to save clothes; Bathroom problems, difficulty voiding/unable to get to bathroom in time; Constipation or diareaha; May need help with personal hygene; Needs help bathing; May need hand rails/bathing bench, etc., a mobile shower head is a good idea, if possible; Weakness or neglect on one side of body, one side more dominant, i.e. drags left or right foot, etc. (Shydragger syndrome); Subject to infections, urinary tract, respiatory tract (pneumonia) etc.; Alien hand, sometimes holds on to things and cannot let go or takes the hand a long time to release; Difficulty concentrating, sometimes seems ‘out of it’.

Phase 3—Some obsessive-compulsive behavior, i.e. fingers “pill rolling”, hands smoothing out imaginary wrinkles on table, etc.; Increased irritability; Increased impatience; May become incontinent of urine and bowel; Increased speech problems, often very difficult to understand; Cannot articulate proper speech sounds; Increased eating problems; More coughing/choking; Increased cognitive problems; Cannot follow stories on TV; Cannot read much, due to vision; Some suffer from ‘sensory overload’; Sleeps much of the day, and all night, too; Instances of ‘restless leg’ syndrome; Limbs and neck may become rigid; May loose ability to support self on legs; Increased falls; Some falls may be close to being described as ‘seizures’; Complete loss of control of arms and legs, with reslutant fall; After fall, will sleep for an hour or so; May not always know whether is injured or not; May not ‘feel’ the injury; Increased coughing and choking; Drooling becomes common, often does not close mouth; Infections may be more frequent; Requires much more help in dressing and with all activities of daily living; Does not speak much, but does enjoy seeing friends and relatives, even though patient may not respond much to them; May have pain in arms or legs; Non-specific pain for no apparent reason, application of ‘heat’ rubs may help; Tylenol may also help.

Phase 4—Unintelligible speech/mumbling; Cannot say words; May go days with out saying anything; Constant drooling; Coughing and choking may become so severe that eating normally is impossible; Dr. may reccomend feeding tube, which requires a surgical procedure to install; May have trouble opening mouth, even for meds; Increased incontinence/constipation problems; Losing interest in daily activities; Sleeps most of the time; Uncomfortable sitting for any length of time, prefers bed; Cannot support self on legs; ‘spaghetti legs’; Body rigid, especially neck area; Little eye movement; Cannot ‘look’ at something; Slow to focus on things in view; Delusions, hallucinations at times; May be disoriented and not know where they are; Pain, but cannot identify the area; Withdrawn, but remains aware of people; Cannot move on own; Needs extensive help for all activities of daily living.

Note: These phases or categories often overlap and are not the same for all patients. Some may have two or three phase 1 problems and one phase 3 problem. Some may never have all of the problems, but most will need extensive help to live out their lives and will need to be made as comfortable as possible for the duration of their illness.

Rasagiline, R(+)-N-propargyl-1-aminoindan, is a potent second generation monoamine oxidase (MAO) B inhibitor (Finberg et al., Pharmacological properties of the anti-Parkinson drug rasagiline; modification of endogenous brain amines, reserpine reversal, serotonergic and dopaminergic behaviours, Neuropharmacology (2002) 43(7):1110-8). Rasagiline Mesylate in a 1 mg tablet is commercially available for the treatment of idiopathic Parkinson's disease as AZILECT® from Teva Pharmaceuticals Industries, Ltd. (Petach Tikva, Israel) and H. Lundbeck A/S (Copenhagen, Denmark). Recent studies have demonstrated that, in addition to its MAO-B inhibitor activity, rasagiline possesses potent neuroprotective activity demonstrated by in vitro and in vivo experiments. Neuroprotection by rasagiline was achieved in animal models of closed head trauma (Huang et al., Neuroprotective effect of rasagiline, a selective monoamine oxidase-B inhibitor, against closed head injury in the mouse, Eur. J. Pharmacol. (1999) 366(2-3):127-35), global focal ischemia (Speiser et al., Studies with rasagiline, a MAO-B inhibitor, in experimental focal ischemia in the rat, J. Neural Transm. (1999) 106(7-8):695-606) and MPTP-induced neurotoxicity (Sage et al. 2001, 2003) as well as transgenic model of amyotrophic lateral sclerosis (Waibel et al., Rasagiline alone and in combination with riluzole prolongs survival in an ALS mouse model, J. Neurol. (2004) 251(9):1080-4) and 6-OHDA model of PD (Blandini et al., Neuroprotective effect of rasagiline in a rodent model of Parkinson's disease, Exp. Neural. (2004) 187(2):455-9). Cell culture experiments have shown that rasagiline potently suppresses apoptotic cell death initiated by mitochondria (Youdim et al., Rasagiline [N-propargyl-1R-(+)-aminoindan], a selective and potent inhibitor of mitochondrial monoamine oxidase B Br. J. Pharmacol. (2001) 132(2):500-6; Akao et al., Mitochondrial permeability transition mediates apoptosis induced by N-methyl(R)salsolinol, an endogenous neurotoxin, and is inhibited by Bcl-2 and rasagiline, N-propargyl-1(R)-aminoindan, J. Neurochem. (2002) 82(4):913-23) by preventing preapoptotic swelling of mitochondria, caspase 3 activation, activation of nuclear PARP-1, translocation of GADPH, and nucleasomal DNA fragmentation (Youdim and Weinstock, Molecular basis of neuroprotective activities of rasagiline and the anti-Alzheimer drug TV3326 [(N-propargyl-(3R)aminoindan-5-YL)-ethyl methyl carbamate], Cell Mol. Neurobiol. (2001) 21(6):555-73; Youdim et al., Amyloid processing and signal transduction properties of antiparkinso-antialzheimer neuroprotective drugs rasagiline and TV3326, Ann. N.Y. Acad. Sci. (2003) 993:378-86; Bar-am et al., Regulation of protein kinase C by the anti-Parkinson drug, MAO-B inhibitor, rasagiline and its derivatives, in vivo, J. Neurochem. (2004) 89(5):1119-25; and Weinreb et al., Neuroprotectoin via pro-survival protein kinase C isoforms associated with Bcl-2 family members, Faseb J. (2004) 18(12):1471-3). Further, rasagiline induces increase of the anti-apoptotic Bcl-2 and Bcl-xL expression parallel to downregulation of pro-apoptotic Bad and Bax (Youdim et al., The essentiality of Bcl-2, PKC and proteasome-ubiquitin complex activations in the neuroprotective-antiapoptotic action of the anti-Parkinson drug, rasagiline, Biochem. Pharmacol. (2003) 66(8):1635-41; Yogev-Falach et al., The importance of propargylamine moiety in the anti-Parkinson drug rasagiline and its derivatives in MAPK-dependent amyloid precursor protein processing, Faseb J. (2003) 17(15):2325-7; Bar-Am et al., supra). Recent evidence from a delayed-start design study in PD has suggested potential disease-modifying efficacy of rasagiline also in a clinical setting (Parkinson Study, G., A controlled, randomized, delayed-start study of rasagiline in early Parkinson disease, Arch. Neurol. (2004) 61(4):561-6).

### EXPERIMENTAL DETAILS

### Example 1

### Clinical Use of Rasagiline for Treatment of PSP Patients

Rasagiline tablets (Azilect®, Teva Pharmaceutical Industries Ltd.) at a dose of 1 mg rasagiline/day (in the form of 1.56 mg rasagiline mesylate) were administered to 16 PSP patients over 12 months and one patient over 9 months. The mean age was 67±8 years (all values are mean±standard deviation). The mean value of the PSP rating scale (PSPRS) was 54±14 points. The duration of the disease was between 4 to 144 months. Eight men and nine women were treated.

TABLE 1
Demographic data:

| | Age | | PSPRS | | duration |
|---|---|---|---|---|---|
| Nr | (years) | sex | (points) | onset: | (months) |
| 1 | 78 | M | 51 | 2003 | 84 |
| 2 | 64 | W | 48 | 2005 | 31 |
| 3 | 63 | M | 72 | 2002 | 35 |
| 4 | 69 | M | 76 | 1995 | 144 |
| 5 | 77 | M | 46 | 2003 | 28 |
| 6 | 59 | W | 41 | 2001 | 72 |
| 7 | 80 | W | 73 | 2004 | 23 |
| 8 | 60 | W | 68 | 2003 | 35 |
| 9 | 68 | W | 64 | 2003 | 45 |
| 10 | 68 | W | 42 | 2004 | 18 |
| 11 | 70 | M | 27 | 2006 | 4 |
| 12 | 68 | W | 49 | 2003 | 37 |
| 13 | 58 | M | 42 | 2006 | 8 |
| 14 | 57 | M | 52 | 2004 | 36 |
| 15 | 68 | W | 69 | 2001 | 72 |
| 16 | 70 | M | 54 | 2002 | 60 |
| 17 | 66 | W | 49 | 2005 | 23 |

The following clinical factors were analyzed:
1. Patients and relatives received a protocol to document the frequency of falls.
2. 12 of the patients were analyzed using posturographic measurements.
3. Depression was evaluated using clinical criteria (DSM-IV).
4. Eye movements were orthoptically evaluated (in some cases an electronystagmogram was performed).
5. Dysarthria was investigated using the Bogenhausener dysarthria scale (BoDys) (ranges from 4=normal until 0=anarthria).
6. Dysphagia was clinically documented and the introduction of a percutaneous feeding tube (PEG) was recorded.
7. Incidence of pneumonia was recorded.
8. Impulse control disorder and hallucinations were recorded.
9. Possible side effects were recorded.
10. Treatment with other medication, other than Rasagiline, was recorded.

### Results

### Patients/Side Effects/Treatment Complications

Ten of the 17 patients were administered rasagiline over the complete observation time of 12 months. One patient had it for only 9 months. Two patients died during the observation time. Patient 9 died of pneumonia one month after the drug treatment (which had been only one month) had been ended. Patient 4 died suddenly 4 months after 8 months of treatement had been ended. Patient 12 had a severe fall after 2 months of drug treatment and suffered from intracranial haemorrhage. During her hospitalization, almost all drug treatment including rasagiline was stopped. The treating physician assessed these events as not related to rasagiline.

Three patients showed side effects which terminated the use of rasagiline. Patient 3 developed bladder disturbances 14 days after initiation of treatment, patient 10 developed headaches 16 days after initiation and patient 16 developed headaches after 27 days of treatment. Rasagiline treatment was terminated in these three patients, and the side effects were completely reversible.

### Falls

Before treatment the patients had a mean of 23±9 falls/week. Table 2 shows the registered frequency of falls of all patients. The reduction of the frequency mainly took place within the first 7 months of treatment.

TABLE 2
Temporal profile of the frequency of falls during treatment

| | | 1 | 2 | 3 | 4 | 5 | 6 | 7 | 8 | 9 | 10 | 11 | 12 |
|---|---|---|---|---|---|---|---|---|---|---|---|---|---|
| | baseline | Month | Months | Months | Months | Months | Months | Months | Months | Months | Months | Months | Months |
| 1 | 17 | 13 | 12 | 12 | 9 | 12 | 13 | 18 | 16 | 15 | 18 | 18 | 20 |
| 2 | 32 | 25 | 24 | 22 | 24 | 22 | 22 | 22 | 29 | 26 | 24 | 28 | 29 |
| 3 | 28 | 22 | | | | | | | | | | | |
| 4 | 34 | 28 | 27 | 28 | 29 | 29 | 25 | 26 | 28 | | | | |
| 5 | 35 | 28 | 26 | 28 | 24 | 26 | 27 | 29 | 30 | 28 | 34 | 36 | 34 |
| 6 | 14 | 12 | | | | | 12 | | | | | | 12 |
| 7 | 20 | 21 | 20 | 22 | 22 | 21 | 19 | 23 | 24 | 24 | 22 | 22 | 22 |
| 8 | 21 | 19 | 19 | 18 | 20 | 20 | 21 | 22 | 22 | 24 | 26 | 28 | 26 |
| 9 | 28 | 28 | 28 | 28 | 27 | 28 | 29 | 29 | 32 | 32 | 30 | | |
| 10 | 5 | 5 | | | | | | | | | | | |
| 11 | 10 | 2 | 2 | 4 | 4 | 6 | 3 | 4 | 6 | 5 | | | |
| 12 | 28 | 24 | 24 | | | | | | | | | | |
| 13 | 12 | 9 | 9 | 9 | 8 | 8 | 9 | 10 | 11 | 8 | 7 | 10 | 10 |
| 14 | 28 | 26 | 26 | 27 | 27 | 28 | 28 | 28 | 30 | 32 | 33 | 32 | 35 |
| 15 | 32 | 32 | 31 | 30 | 32 | 32 | 32 | 30 | 32 | 33 | 34 | 34 | 36 |
| 16 | 27 | 22 | | | | | | | | | | | |
| 17 | 21 | 21 | 21 | 22 | 22 | 19 | 19 | 22 | 22 | 24 | 23 | 25 | 22 |
| MW | 23.059 | 19.824 | 20.692 | 20.833 | 20.667 | 20.917 | 19.923 | 21.917 | 23.5 | 22.818 | 25.1 | 25.889 | 24.6 |
| Stdv | 8.9824 | 8.6836 | 8.3804 | 8.4728 | 8.9375 | 8.4687 | 8.6261 | 7.9711 | 8.544 | 9.548 | 8.4255 | 8.2529 | 9.1554 |

### Depression

A total of 16 patients had signs of a depressed mood at the beginning of the evaluation. Three of them were receiving antidepressant medication (Fluoxetin, Cipramil, Cipralex) at the initiation of the trial. This medication was ended at the time rasagiline was initiated because of possible drug interactions. However, none of these patients reported a depressed mood. There was no new development of depression during treatment with rasagiline, as none of the patients receiving rasagiline had to be treated with antidepressants.

### Dysphagia

Two patients already had a PEG at the initiation of the trial. However, during the observation phase none of the other patients required a PEG, which may indicate a slow progression of dysphagia.

### Dysarthria

During the observation time there was a slight improvement in dysarthria in 10 patients from a mean of 2.5±0.5 to 2.7±0.5 on the BoDys scale.

### Eye Movements

No effect of rasagiline on eye movements was been observed.

### Pneumonia

Two patients developed pneumonia during the observation time. Patient 9 developed pneumonia after 9 months of treatment and died within 3 weeks (at the onset of pneumonia the treatment with rasagiline was already stopped). Patient 4 developed pneumonia within two weeks of treatment then recovered. He suddenly died 7 months later.

### Impulse Control Disorder/Hallucinations

In the treated patient group at the time of initiation of rasagiline treatment, one patient suffered from an impulse control disorder which was not influenced by treatment with rasagiline. There were no hallucinations at the onset of treatment and no patients developed hallucinations during the observation time.

### Additional Medications

The additional medications which the patients were taking are shown in Table 3.

TABLE 3
Additional Medication

| Medication | Frequency |
|---|---|
| L-Dopa | 7 |
| Amantadine | 8 |
| Cabergoline | 1 |
| Baclofen | 3 |
| Amitriptyline | 3 |
| riluzole | 2 |
| Domperidone | 3 |
| Trospium | 3 |
| Aspirin | 1 |
| Zopiclone | 1 |

### Analysis

In the group of patients wherein rasagiline was administered, improvement in postural instability was evident within the first 7 months. This can be seen by a reduction in the mean number of weekly falls during the course of this period. This is an important finding since these patients are severely affected by frequent falls.

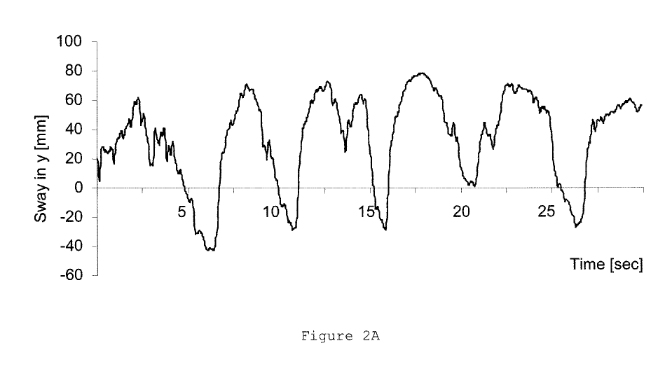

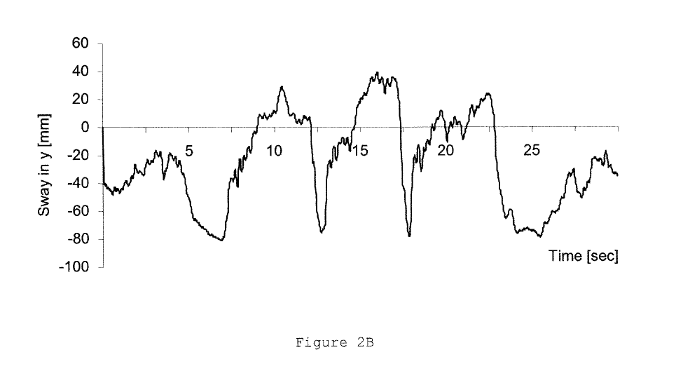

In addition, posturographic measurement (as in FIG. 2) shows an improvement over when comparing a patient before treatment and after 6 months of treatment with rasagiline.

Additionally, there was no onset of depression which is a common symptom, usually occurring early in the disease. Patients that were treated with anti-depressant prior to the rasagiline treatment did not require anti-depressant treatment during the course of the rasagiline treatment. Treatment with rasagiline did not induce hallucinations.

Interestingly, the frequency of pneumonia was very low during treatment with rasagiline. This is an important yet unexplained finding since respiratory parameters are markedly reduced in these patients because of the axial rigor.

Side effects which possibly are linked to the drug have been seen in three patients. After discontinuing the treatment they were completely reversible. Two patients developed headache and one bladder disturbances.

This documentation of the treatment effect of rasagiline shows that this drug is suitable for treatment of PSP.

### Example 2

### A Randomized, Monocenter, Double-Blind, Placebo-Controlled, Parallel-Group, Phase IIb Study to Assess the Efficacy, Tolerability and Safety of Rasagiline in Subjects with Progressive Supranuclear Palsy

A clinical trial is performed according to the following guidelines:

| Study | Name: Azilect ® Tablets | |
|---|---|---|
| Medication, Dose | Generic name: Rasagiline | |
| and Mode of | Dose: 1 mg/day | |
| Application | Mode of Application: oral | |
| | Duration of Treatment: 1 year | |
| Comparative | Placebo: manufactured by the same company | |
| Drug, Dose and | (tablet without active compound) | |
| Mode of | Dose: not applicable | |
| Application | Mode of Application: oral | |
| | Duration of Treatment: 1 year | |
| Study Population | Male and female patients with PSP according to | |
| | the NNIPPS criteria, early stage (PSP staging ≦II), | |
| | PSP Rating Scale (PSPRSC) <29 | |
| Study Design | Monocenter, prospective, randomised, double | |
| | blind, placebo controlled. | |
| | Comparing placebo with 1 mg rasagiline as | |
| | therapy in 112 enrolled PSP patients. For entry | |
| | the patients are allowed to be on L-Dopa therapy | |
| | but the dose must be stable for the last 3 | |
| | months. After screened for inclusion the | |
| | patients are randomly assigned to treatment with | |
| | either placebo or rasagiline according to their | |
| | stratum. | |
| | The trial requires an initial screening visit | |
| | (V0), a baseline visit (V1), 3 subsequent visits | |
| | (V2-V4) and a final visit (V5). Additionally 4 | |
| | telephone contacts are scheduled. Patients | |
| | receive a diary. | |
| | A follow-up phase is planned, starts after FPLV. | |
| Study Objectives | Primary Objective | |
| | To assess the efficacy of rasagiline using the | |
| | PSP rating scale (PSPRS), aiming at a 33% | |
| | reduction of the reported deterioration (Golbe | |
| | L I, (June 2007), Ohman-Strickland P A. Brain; | |
| | 130(Pt 6): 1552-65; (Apr. 2, 2007) Epub; A | |
| | clinical rating scale for progressive | |
| | supranuclear palsy), i.e. a mean yearly increase | |
| | of 6.5 instead of 9.7. | |
| | To assess the need for additional L-Dopa therapy | |
| | or the need to increase the dose of L-Dopa | |
| | during the trial. | |
| | Secondary Objective | |
| | Reduction of gait disturbances and postural | |
| | stability (as documented with posturographic | |
| | measurement). | |
| | Clinical safety and tolerability is assessed by | |
| | findings of physical and neurological | |
| | examination, laboratory variables, adverse | |
| | events incidence, vital signs, ECG, assessment | |
| | of survival time | |
| | Number of Subjects (%) who discontinue the study | |
| | Number of Subjects (%) who discontinue the study | |
| | due to AEs | |
| | Assessment of survival time | |
| | Additional endpoints: Secondary efficacy | |
| | variables also include incidence of dysphagia, | |
| | gastrostomia, depression and pneumonia | |
| Study Endpoints | Primary Efficacy Endpoint: | |
| | The primary outcome measure is the integral | |
| | of the PSPRS changes from baseline over | |
| | time measured during visits at 3, 6, 9, 12 | |
| | months. | |
| | The need for additional L-Dopa therapy or | |
| | the need to increase the dose of L-Dopa | |
| | during the trial. | |
| | Secondary Efficacy Endpoints: | |
| | Development of gait disturbance/postural | |
| | instability from baseline in posturographic | |
| | measurement and corresponding costs | |
| | Change from baseline in pulmonary function | |
| | Time to pneumonia and corresponding costs | |
| | Time to gastrostomia and corresponding costs | |
| | Time to develop/progress of dementia and | |
| | corresponding costs | |
| | Change from baseline in depression scale | |
| | and corresponding costs | |
| | Changes from baseline in individual quality | |
| | of life | |
| | Changes from baseline in disease-related | |
| | quality of life | |
| | Time to death | |
| | Secondary Endpoints for Safety and Tolerability: | |
| | Tolerability | |
| | Number of subjects (%) who discontinue the | |
| | study | |
| | Number of subjects (%) who discontinue the | |
| | study due to AEs | |
| | Safety | |
| | AE incidence | |
| | Safety laboratory values (blood cell count, | |
| | ASAT, ALAT, creatinine) | |
| | Vital signs | |
| | ECG | |
| | Physical and neurological examination | |
| Patient Number | Total number of 112 patients. 56 patients are | |
| | randomized to treatment with 1 mg Rasagiline and | |
| | 56 patients to treatment with placebo. | |
| Inclusion Criteria | Subjects must meet all Inclusion criteria to be | |
| | eligible: | |
| | 1. | Clinical signs of PSP. |
| | | Diagnosis is made for patients with |
| | | clinical probable PSP (Litvan I, Agid Y, |
| | | Jankovic J, Goetz C, Brandel J P, Lai E C, |
| | | Wenning G, D'Olhaberriague L, Verny M, |
| | | Chaudhuri K R, McKee A, Jellinger K, Bartko |
| | | J J, Mangone C A, Pearce R K; (1996), Accuracy |
| | | of clinical criteria for the diagnosis of |
| | | progressive supranuclear palsy (Steele- |
| | | Richardson-Olszewski syndrome), Neurology, |
| | | 46: 922-30). |
| | | Patients are included with PSP stage ≦II |
| | | (Golbe L I, (1997), A clinical rating scale |
| | | and staging system for progressive |
| | | supranuclear palsy, Neurology; |
| | | 48(Suppl): A326.), at least with a PSPRS <29 |
| | | (Golbe L I, (June 2007), Ohman-Strickland |
| | | P A. Brain; 130(Pt 6): 1552-65; (Apr. 2, |
| | | 2007) Epub; A clinical rating scale for |
| | | progressive supranuclear palsy) and |
| | | according to the diagnostic criteria resumed |
| | | after the NNIPPS trial (Bensimon G, |
| | | Ludolph A, Agid Y, Vidailhet M, Payan C, |
| | | Leigh P N; (January 2009) NNIPPS Study |
| | | Group. Brain; 132(Pt 1): 156-71. Riluzole |
| | | treatment, survival and diagnostic criteria |
| | | in Parkinson plus disorders: the NNIPPS |
| | | study). |
| | 2. | Patients, male or female, aged 50 to 80 |
| | | years |
| | 3. | Subjects whose clinical condition at the |
| | | time of enrolment do not require L-DOPA or |
| | | require a low [≦400 mg/day] stable dose |
| | | of L-DOPA for at least 3 months prior to |
| | | study entry. |
| | 4. | Capability and willingness to give written |
| | | signed and dated informed consent document |
| | | indicating that the subject (or a legally |
| | | acceptable representative) has been |
| | | informed of all pertinent aspects of the |
| | | study. |
| Exclusion | 1. | No clinically probable PSP |
| Criteria | 2. | No written informed consent possible |
| | 3. | Age >80 or <50 years |
| | 4. | Dementia (MMSE ≦24) |
| | 5. | Subjects with clinically significant |
| | | psychiatric illness, including major |
| | | depression. |
| | 6. | Subjects who have taken any experimental |
| | | drugs within 60 days prior to baseline. |
| | 7. | Subjects who have used sympathomimetics |
| | | (including over-the-counter remedies - |
| | | nasal or oral), dextromethorphan, pethidine |
| | | or St. John's wort within 7 days prior to |
| | | baseline. |
| | 8. | Loss of postural reflexes (no independent |
| | | walking possible, inability to stand |
| | | unassisted, wheelchair-bound) |
| | 9. | Feeding tube/recommendation for a feeding tube |
| | 10. | Unintelligible speech |
| | 11. | History of brain disease (e.g. repeated |
| | | strokes, cerebral tumour, hydrocephalus) |
| | 12. | MPTP exposure |
| | 13. | Oculogyric crisis |
| | 14. | Early severe autonomic failure |
| | 15. | Systemic disorder affecting the brain |
| | 16. | Women who are not postmenopausal or |
| | | surgically sterilized. |
| | 17. | Known history of hypersensitivity to the |
| | | investigational drug or to drugs with a |
| | | similar chemical structure |
| | 18. | Subjects who have used antidepressants, |
| | | including selective serotonin re-uptake |
| | | inhibitors, tricyclic and tetracyclic |
| | | antidepressants (except amitriptyline <=50 |
| | | mg/day, trazodone <=100 mg/day, |
| | | citalopram <=20 mg/day, sertaline <=100 |
| | | mg/day and paroxetine <=30 mg/day, |
| | | escitalopram <=10 mg/day) within 42 days |
| | | prior to baseline. |
| | 19. | Subjects who have used any drugs known to |
| | | have been involved in a drug interaction |
| | | via inhibition of hepatic CYP 1A2 within 30 |
| | | days prior to baseline (cimetidine, |
| | | ciprofloxacin, clarithromycin, enoxacin, |
| | | erythromycin, fluvoxamine, isoniazide, |
| | | nalidixic acid, norfloxacin, |
| | | troleandomycin, zileuton) |
| | 20. | Subjects who have used MAO inhibitors |
| | | including reserpine and methyldopa or |
| | | coenzyme Q10 within three months prior to |
| | | baseline |
| | 21. | Anti-emetic or antipsychotic medication |
| | | with central dopamine antagonist activity |
| | | (except quetiapine fumarate) within six |
| | | months prior to baseline. |
| | 22. | Participation in a clinical trial within |
| | | the last 30 days prior to study start. |
| | 23. | Unstable antiparkinsonian medication within |
| | | 30 days before baseline |
| | 24. | Previous use of rasagiline or selegiline. |
| | 25. | Subjects who have a clinically significant |
| | | or unstable medical or surgical condition |
| | | that may preclude safe and complete study |
| | | participation (based on the investigator's |
| | | judgment). Such conditions might include |
| | | cardiovascular, vascular diseases, |
| | | pulmonary, hepatic impairment (Child-Pugh |
| | | score >5), renal, or metabolic diseases or |
| | | malignancies as determined by medical |
| | | history, physical examination, laboratory |
| | | tests, chest x-ray or ECG. |

| | |
|---|---|
| Study Procedures | every 3 months: Neurological tests, diary, |
| | posturographic measurement, lung function, |
| | depression scale, monitoring of medication, |
| | side effects, self assessment |
| | Screening visit (V0) |
| | Medical history |
| | Informed consent |
| | Neurological examination |
| | Golbe score, PSP stageing |
| | Visit 1 - Baseline visit (V1) |
| | Physical examination |
| | Posturographic measurement |
| | Schwab and England score, UPDRS part II. |
| | Neurological examination including Golbe |
| | Score, PSP staging system, UPDRS part III, |
| | Mini Mental State Evaluation, Frontal |
| | Assessment Battery and UPDRS part I. |
| | Lung function (spirometric evaluation) |
| | ECG |
| | Montgomery-Åsberg Depression Rating Scale |
| | PSP-QoL scale |
| | SmiLE |
| | Blood test |
| | Visit 2-4 - Control visits (V2, V3, V4) |
| | Physical examination |
| | Patient diary (falls) |
| | Posturographic measurement |
| | Schwab and England score, UPDRS part II. |
| | Neurological examination including Golbe |
| | Score, PSP stageing system, UPDRS part III, |
| | Mini Mental State Evaluation, Frontal |
| | Assessment Battery and UPDRS part I |
| | Adverse events and changes in concomitant |
| | medication |
| | Lung function |
| | ECG |
| | Montgomery-Asberg Depression Rating Scale |
| | Visit 5 - Final Visit (V5) |
| | Physical examination |
| | Patient diary (falls) |
| | Posturographic measurement |
| | Schwab and England score, UPDRS part II. |
| | Neurological examination including Golbe |
| | Score, PSP stageing system, UPDRS part III, |
| | Mini Mental State Evaluation, Frontal |
| | Assessment Battery and UPDRS part I |
| | Adverse events and changes in concomitant |
| | medication |
| | Lung function |
| | ECG |
| | Montgomery-Asberg Depression Rating Scale |
| | PSP-QoL |
| | SmiLE |
| | 4 Telephone contacts (T1-T4): |
| | First call is 4 weeks after beginning of study |
| | treatment. The following calls will be after 4, |
| | 7 and 10 months. Within every telephone contact |
| | the patient is asked specifically for the |
| | following issues: |
| | Intake of trial medication - problems? |
| | SAEs, AEs? |
| | Concomittant medication, changes? |
| | Any motor function impairment? |
| | weight |
| Study Specific | Clinical neurological and physical |
| Measurements | Investigation |
| | Posturographic testing |
| | Spirometric measured lung function |
| Statistical | Primary Statistical Analysis: |
| Rationale | Nonparametric comparison of the integrals of |
| | PSPRS changes from baseline over time using the |
| | Mann-Whitney U test |
| | Secondary Endpoints: |
| | Exploratory analyses of the secondary endpoints |
| | outlined above, using appropriate tests |
| | Safety Analysis: |
| | Exploratory comparison of discontinuation rates |
| | and adverse event rates using Fisher's exact tests |
| Time Schedule | Per Patient: |
| | The screening period is 12 months and the |
| | duration of treatment is 12 months resulting |
| | in duration of the trial of about 24 months. |
| | An extra 12-24 months follow-up treatment |
| | after the interventional period is planned for |
| | completing the study. |
| | Study duration: |
| | Recruiting Period: 12 months |
| | Planned Start Date (FPFV): September 2009 |
| | Planned End Date (LPLV): August 2011 |

### CONCLUSIONS

Rasagiline was effective in reducing the number of falls for patients with Progressive Supranuclear Palsy.

Rasagiline was effective in reducing or eliminating depression for patients with Progressive Supranuclear Palsy.

Rasagiline was effective in improving or slowing progression of dysphagia for patients with Progressive Supranuclear Palsy.

Rasagiline, 1 mg/day is effective in treating patients with Progressive Supranuclear Palsy, measured by a 33% reduction of the reported deteroration.

Rasagiline, 1 mg/day is safe and effective in reducing gait disturbances and in enhancing postural stability in patients with Progressive Supranuclear Palsy.

1. A method of treating a human subject suffering from Progressive Supranuclear Palsy, comprising administering to the subject an amount of R(+)-N-propargyl-1-aminoindan or a pharmaceutically acceptable salt thereof effective to treat the subject.

2. A method of alleviating a symptom of Progressive Supranuclear Palsy in a human subject afflicted with Progressive Supranuclear Palsy comprising administering to the subject an amount of R(+)-N-propargyl-1-aminoindan or a pharmaceutically acceptable salt thereof effective to alleviate the symptom of Progressive Supranuclear Palsy in the subject.

3. The method of claim 2, wherein the symptom of Progressive Supranuclear Palsy is postural instability, frequent falls, visual disturbances, speech disturbances, ataxia, dysphagia, pneumonia or depression.

4. The method of claim 1 wherein the amount of R(+)-N-propargyl-1-aminoindan or of the pharmaceutically acceptable salt thereof is from 0.01 mg to 20 mg per day.

5. The method of claim 4 wherein the amount of R(+)-N-propargyl-1-aminoindan or of the pharmaceutically acceptable salt thereof is from 0.5 mg to 5 mg per day.

6. The method of claim 4 wherein the amount of R(+)-N-propargyl-1-aminoindan or of the pharmaceutically acceptable salt thereof is 2 mg per day.

7. The method of claim 4 wherein the amount of R(+)-N-propargyl-1-aminoindan or of the pharmaceutically acceptable salt thereof is 1 mg per day.

8. The method of claim 4 wherein the amount of R(+)-N-propargyl-1-aminoindan or of the pharmaceutically acceptable salt thereof is 0.5 mg per day.

9. The method claim 1 wherein the administration is of the pharmaceutically acceptable salt of R(+)-N-propargyl-1-aminoindan.

10. The method of claim 9 wherein the pharmaceutically acceptable salt is esylate, mesylate, sulphate, citrate or tartrate.

11. The method of claim 10 wherein the pharmaceutically acceptable salt is mesylate.

12. The method of claim 11 wherein the amount of R(+)-N-propargyl-1-aminoindan mesylate is 1.56 mg per day.

13. The method of claim 1 wherein the administration is oral, parenteral, rectal or transdermal.

14. (canceled)

15. (canceled)
